(19) United States
(12) Patent Application Publication
(10) Pub. No.: US 2026/0334780 A1
(43) Pub. Date: Oct. 29, 2026

(54) AUTOMATED TROCAR PLACEMENT GUIDANCE FOR SURGICAL PROCEDURES

(71) Applicant: Alcon Inc., Fribourg (CH)

(72) Inventors: Nikhil SCARIA, Fort Worth, TX (US); Lu YIN, Fort Worth, TX (US); Vignesh SURESH, Fort Worth, TX (US); Ramesh SARANGAPANI, Fort Worth, TX (US); Joseph WEATHERBEE, Fort Worth, TX (US); Zhuoran WU, Fort Worth, TX (US); Aakash NAGARAJAN, Fort Worth, TX (US); Sinchan BHATTACHARYA, Fort Worth, TX (US)

(21) Appl. No.: 19/657,984

(22) Filed: Apr. 24, 2026

Related U.S. Application Data
(60) Provisional application No. 63/794,975, filed on Apr. 25, 2025.

(51) Int. Cl. A61B 3/13; A61B 3/00; A61B 3/107; A61B 34/20

(52) U.S. Cl. CPC A61B 3/13; A61B 3/0025; A61B 3/0041; A61B 3/107; A61B 34/20; A61B 2034/2065

(57) ABSTRACT

Certain embodiments provide an optical imaging system including an ophthalmic microscope and an image processing system. The image processing system includes a camera attached to the ophthalmic microscope, a display, and a processing system. The processing system includes a processor that is configured to receive a video stream from the camera. The video stream includes a sequence of video frames, and each video frame includes image data of an eye of a patient. For each video frame, the processor is configured to generate a segmentation mask identifying a limbus of the eye, determine geometric characteristics of the limbus, determine a safe insertion region located posterior to the limbus, determine one or more trocar insertion locations within the safe insertion region, generate an overlay identifying the one or more trocar insertion locations, and present the overlay with the image data on the display.

### CROSS-REFERENCE TO RELATED APPLICATIONS

This application claims the benefit of U.S. Provisional Application Ser. No. 63/794,975 (filed on Apr. 25, 2025), the content of which is incorporated by reference herein in its entirety.

### BACKGROUND

A variety of diseases or conditions associated with an eye may be treated through ophthalmic surgical procedures. Examples of ophthalmic surgical procedures include vitreoretinal surgery, cataract surgery, glaucoma surgery, laser eye surgery (e.g., Laser-Assisted In Situ Keratomileusis or LASIK), and the like. For example, vitreoretinal surgery is a type of eye surgery that treats problems with the retina or the vitreous and may be performed for treating conditions such as diabetic traction retinal detachment, diabetic vitreous hemorrhage, macular hole, retinal detachment, epimacular membrane, and many other ophthalmic conditions. As another example, cataract surgery is a type of eye surgery that replaces a clouded natural lens in the eye of a patient with an artificial intraocular lens (IOL) to restore clear vision.

### BRIEF DESCRIPTION OF THE DRAWINGS

FIG. 1 depicts a cross-sectional diagram of an eye of a patient during an ophthalmic surgery, in accordance with certain embodiments of the present disclosure.

FIGS. 2A, 2B, 2C depict a process to determine a trocar insertion location, in accordance with an embodiment of the present disclosure.

FIG. 3A depicts an example optical imaging system, in accordance with certain embodiments of the present disclosure.

FIG. 3B presents a diagram of the example optical imaging system depicted in FIG. 3A, in accordance with certain embodiments of the present disclosure.

FIG. 3C depicts a left side view of the example stereoscopic microscope depicted in FIG. 3A, in accordance with certain embodiments of the present disclosure.

FIG. 4A depicts another example optical imaging system, in accordance with certain embodiments of the present disclosure.

FIG. 4B presents a diagram of the example optical imaging system depicted in FIG. 4A, in accordance with certain embodiments of the present disclosure.

FIG. 4C depicts a left side view of the example monoscopic microscope depicted in FIG. 4A, in accordance with certain embodiments of the present disclosure.

FIG. 5 depicts a data flow diagram for providing visualization guidance for trocar placement during ophthalmic surgical procedures, in accordance with embodiments of the present disclosure.

FIG. 6 depicts a 3D view of an eye, in accordance with an embodiment of the present disclosure.

FIG. 7 depicts a 2D cross-sectional view of the eye of FIG. 6 in the YZ plane, in accordance with embodiments of the present disclosure.

FIG. 8 depicts a 2D projection of an overlay and the eye outline onto the XY plane, in accordance with embodiments of the present disclosure.

FIG. 9 depicts a three-trocar configuration of trocar insertion locations, in accordance with embodiments of the present disclosure.

### DETAILED DESCRIPTION

Embodiments of the present disclosure will now be described with reference to the drawing figures, in which like reference numerals refer to like parts throughout.

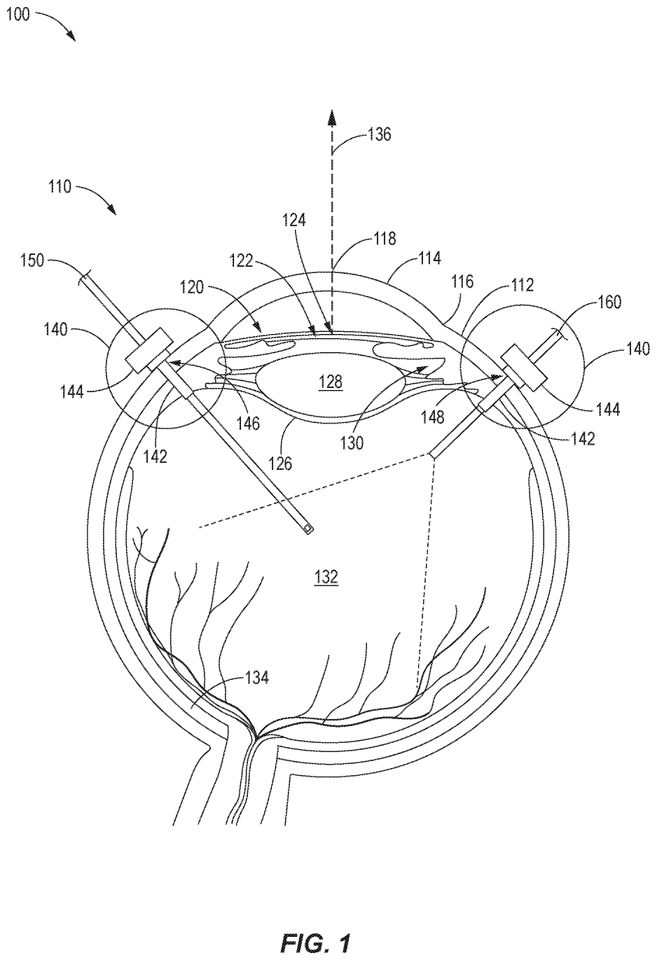

FIG. 1 depicts a cross-sectional diagram 100 of an eye 110 of a patient during an ophthalmic surgery, in accordance with certain embodiments of the present disclosure.

Various anatomical structures of the eye 110 are depicted, including the sclera 112, the cornea 114, the limbus 116 (the junction between the sclera 112 and the cornea 114), the corneal apex 118, the iris 120, the pupil 122, the pupil center 124, the lens capsule 126, the crystalline lens 128, the ciliary body 130, the posterior chamber 132 containing the vitreous humor, and the retina 134. The corneal apex 118 represents the most anterior point of the cornea 114 and typically corresponds to the region of greatest curvature (steepest portion) of the anterior corneal surface.

The visual axis 136 of the eye 110 is illustrated as a reference line passing through the corneal apex 118 and the pupil center 124. This axis approximates the path of light rays that achieve optimal visual acuity when the eye fixates on a distant object, playing a critical role in determining the eye's precise focusing and alignment for high-resolution central vision during fixation tasks.

As illustrated in FIG. 1, a vitrectomy procedure typically involves the precise insertion of one or more surgical tools, such as a vitrector 150, a light probe 160, and so on, into the eye 110 through a trocar cannula 142. A trocar assembly 140 includes a trocar, a trocar cannula 142, and a valve seal 144. The trocar cannula 142 is a hollow tube that is inserted into the eye 110 through an incision created by the trocar. The trocar is a removable sharp or blunt obturator that is disposed within and extends beyond the distal end of the trocar cannula 142. The trocar is withdrawn from the trocar cannula 142 after the trocar cannula 142 is inserted into the eye 110.

In particular, during insertion of the trocar assembly into the sclera 112, the trocar creates a self-sealing sclerotomy. Once the trocar is withdrawn, the trocar cannula 142 remains in place, providing a stable conduit for the introduction and manipulation of microsurgical instruments, such as those having gauges of 23, 25, or 27. The trocar cannula 142 is configured to minimize vitreous traction during instrument passage. The valve seal 144 is positioned at the proximal end of the trocar cannula 142, which substantially prevents the egress of intraocular fluid or gas, thereby helping to maintain intraocular pressure during repeated insertion and removal of instruments.

Generally, the limbus 116 serves as an anatomical landmark for the locations of the trocar cannulas, referred to herein as trocar insertion locations. For safe access to the vitreous within the posterior chamber 132, surgeons target the pars plana region, which is a band just behind the limbus 116. In adult eyes, trocar cannulas 142 are typically inserted about 3.5-4 mm posterior to the limbus 116 (phakic eyes), or 3-3.5 mm for pseudophakic eyes. In infant eyes (smaller globes), this distance can be as little as ~1.5 mm. FIG. 1, illustrates two trocar insertion locations 146, 148 for the trocar cannulas 142.

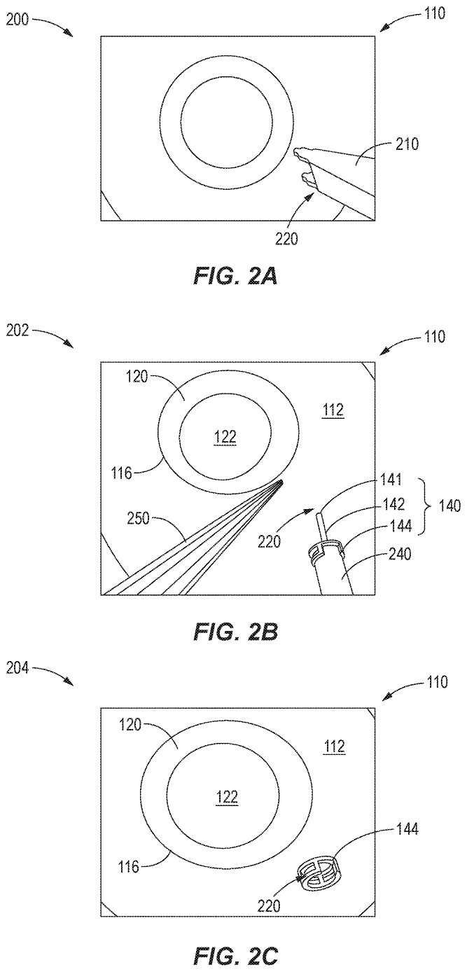

FIGS. 2A, 2B, 2C depict images 200, 202, 204 (respectively) of an eye 110 that illustrate various stages of a manual process to determine a trocar insertion location. The sclera 112, the limbus 116, the iris 120, and the pupil 122 are shown.

As illustrated in FIG. 2A, the first step involves manually positioning a measuring instrument 210 directly against the sclera 112 to establish the precise radial distance from an anatomical landmark, such as the limbus 116, to the intended trocar insertion location, such as the trocar insertion location 220. The measuring instrument 210 is aligned and held steady to obtain an accurate measurement, typically in the range of 3.5 mm to 4.0 mm posterior to the limbus 116 depending on the patient's age and lens status. This measurement step accounts for individual anatomical variations and ensures that subsequent trocar placement will provide safe access to the posterior chamber 132 while avoiding damage to adjacent structures. The measuring instrument 210 forms the foundation for the marking and insertion phases.

The second step involves manually marking the previously measured trocar insertion location 220 on the sclera 112 with a surgical marking instrument (e.g., which may be the measuring instrument 210 itself). The surgical marking instrument applies a temporary yet clearly visible indicator—such as an indentation, dye, or ink—at the exact location determined by the measuring instrument 210. This marking step translates the quantitative measurement into a physical reference that remains identifiable under microscopic visualization throughout the procedure. Precise execution of the marking is critical to preserve the accuracy of the initial measurement and to prevent misalignment during trocar insertion.

As depicted in FIG. 2B, the third step comprises manually advancing the trocar 141 and trocar cannula 142 of the trocar assembly 140 through the sclera 112 precisely at the marked trocar insertion location 220 using a trocar insertion probe 240 while the forceps 250 stabilize the surrounding tissue. The trocar 141 and the trocar cannula 142 are typically introduced at a controlled angle (initially oblique to create a self-sealing tunnel) and then redirected perpendicularly until the trocar cannula 142 is securely seated and the desired intravitreal depth is achieved. Once positioned, the trocar cannula 142 and the valve seal 144 establish a stable access port for infusion lines and surgical instruments. This final insertion step completes the preparatory access while maintaining ocular integrity and enabling the surgeon to proceed with the primary intervention.

FIG. 2C depicts the trocar assembly 140 fully inserted into the eye 110 at the trocar insertion location 220 (only the valve seal 144 is visible).

### Technical Problems

As described in relation to FIGS. 2A-2C, known trocar placement techniques rely on manual identification of anatomical landmarks and manual measurement of insertion locations during ophthalmic surgery. These approaches require the surgeon to position a measuring instrument relative to the limbus, determine a desired offset distance, mark the sclera using a surgical marking instrument, and subsequently insert the trocar at the marked location.

One limitation arises from the manual measurement process. Positioning a measuring instrument against the eye requires additional manipulation of the ocular surface and may require repeated adjustments to obtain an acceptable measurement. Because these steps are performed intraoperatively, they can increase procedure time and interrupt surgical workflow.

Manual measurement is also subject to variability. The resulting measurement may depend on factors such as instrument positioning, eye stability, and visibility of the limbus. Small deviations in measurement or marking can result in insertion locations that differ from the intended anatomical region.

Additionally, known measuring instruments, such as surgical calipers, measuring gauges, and so on, typically provide fixed measurement distances and do not account for variations in patient-specific anatomy. Differences in globe size or anatomical geometry may therefore require the surgeon to adjust measurements based on experience rather than objective guidance.

The use of measuring instruments and surgical marking instruments further increases procedural complexity. These instruments must be introduced, manipulated, and maintained within the surgical field, which can increase workflow burden and require additional sterilized tools.

Further, existing surgical visualization, microscope, and imaging systems are not capable of providing image guidance to assist in this respect. For example, existing surgical visualization, microscope, and imaging systems are generally configured to acquire and display images of the operative field, but they are not configured to computationally interpret those images in a manner that produces, in real-time, anatomically registered trocar-placement guidance during a live ophthalmic procedure. In particular, such systems are not configured to receive microscope image data, detect relevant ocular anatomy from the live video stream, derive a patient-specific insertion zone from the detected anatomy, and generate a graphical overlay that remains spatially aligned with the eye as the eye, microscope, focus, magnification, or field of view changes during surgery. As a result, even when a microscope provides high-quality visualization, the system itself does not automatically convert image content into actionable insertion guidance and instead leaves trocar location determination to separate manual judgment.

Existing systems are further limited by insufficient capability to perform reliable landmark detection under intraoperative conditions, including variable illumination, specular reflections, partial occlusion by instruments, tissue motion, and anatomical variability across different patients. They also generally lack the registration capability needed to update guidance in real time so that an indicated incision location or safe insertion region continues to correspond to the correct anatomical location in image space throughout the procedure.

Accordingly, a technical improvement is needed in microscope-integrated surgical imaging systems to automatically detect ocular anatomical features from live image data, determine one or more patient-specific trocar insertion locations and/or a safe insertion region based on those detected features, and render, in real-time, an overlaid guidance output that is dynamically updated and maintained in registration with the underlying ocular anatomy for intraoperative use.

### Technical Solutions

Embodiments of the present disclosure advantageously provide systems and methods that provide integrated, precise, real-time visualization guidance for trocar placement during ophthalmic surgical procedures, such as vitreoretinal procedures. The visualization guidance does not rely on measuring instruments, surgical marking instruments, or manual techniques, and provides patient-specific guidance across different patients and ophthalmic surgical procedures.

In certain embodiments, an optical imaging system provides visualization guidance as an overlay to a live image of the patient's eye that is presented on a display of the optical imaging system. The overlay includes a safe insertion region and one or more trocar insertion locations that lie within the safe insertion region. The safe insertion region includes an upper boundary and a lower boundary. The upper boundary is the boundary of the limbus of the eye, while the lower boundary is located at a predetermined distance posterior to the limbus.

The overlay identifies the upper boundary and the lower boundary using one type of graphical element, such as a contour line, a boundary line, and so on, and identifies the trocar insertion locations using another type of graphical element, such as circles, squares, crosses, diamonds, and so on. Color may also be used to differentiate the graphical elements, such as a blue line for the upper boundary line, a green line for the lower boundary line, yellow circles for the trocar insertion locations, and so on. The overlay identifies the safe insertion region so that the surgeon may adjust the trocar insertion locations within the safe insertion region based on preference, anatomical features of the sclera (such as blood vessels), and so on.

The optical imaging system processes the image data contained within each frame of a live video stream provided during the surgical procedure, and generates the visualization guidance for each video frame in real time based on the image data. The visualization guidance adapts to changes in the image data, such as movement of the eye or repositioning of the surgical microscope, such that the overlay remains aligned with anatomical features of the eye.

The visualization guidance may be controlled via a user interface presented on a display of the optical imaging system. The surgeon or surgical staff may enable or disable the visualization guidance, select a trocar configuration for a particular ophthalmic surgical procedure, and adjust parameters used to determine the trocar insertion locations. For example, the optical imaging system may support a three-trocar configuration, a four-trocar configuration, and so on. The trocar insertion locations may correspond to predetermined angular positions around the eye and, in some embodiments, may be adjusted based on surgical preferences or procedural requirements.

In certain embodiments, biometric measurements (e.g., pre-operative measurements) of the eye obtained prior to ophthalmic surgery may be incorporated into the determination of the safe insertion region and the trocar insertion locations. The biometric measurements may be provided automatically from other diagnostic systems or entered via the user interface, which adapts the visualization guidance to the patient's ocular geometry.

The visualization guidance also supports surgical planning and training. For example, the safe insertion region and the trocar insertion locations displayed by the overlay provide visual feedback that assists surgeons in confirming trocar placement decisions and supports the training of less experienced medical professionals.

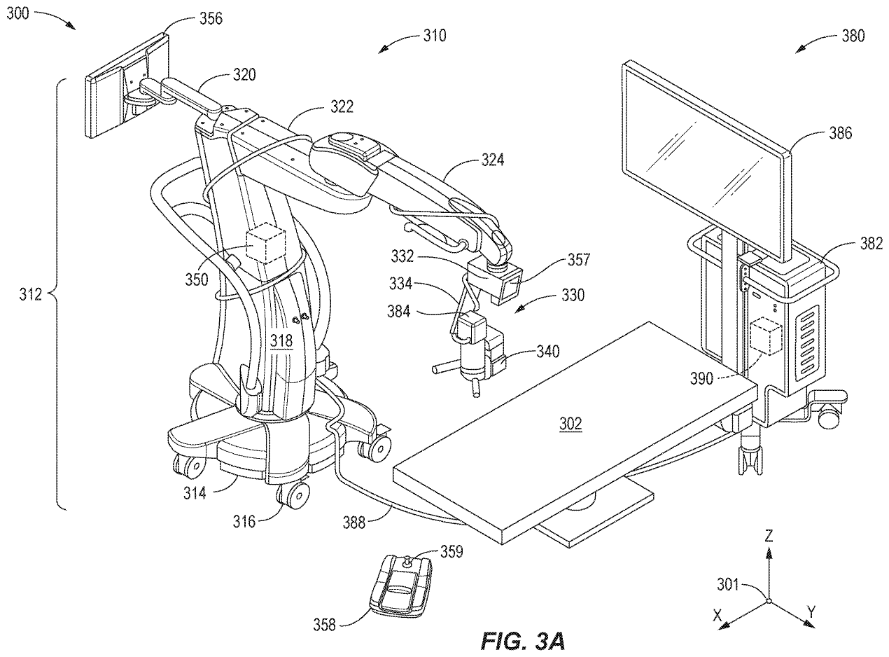

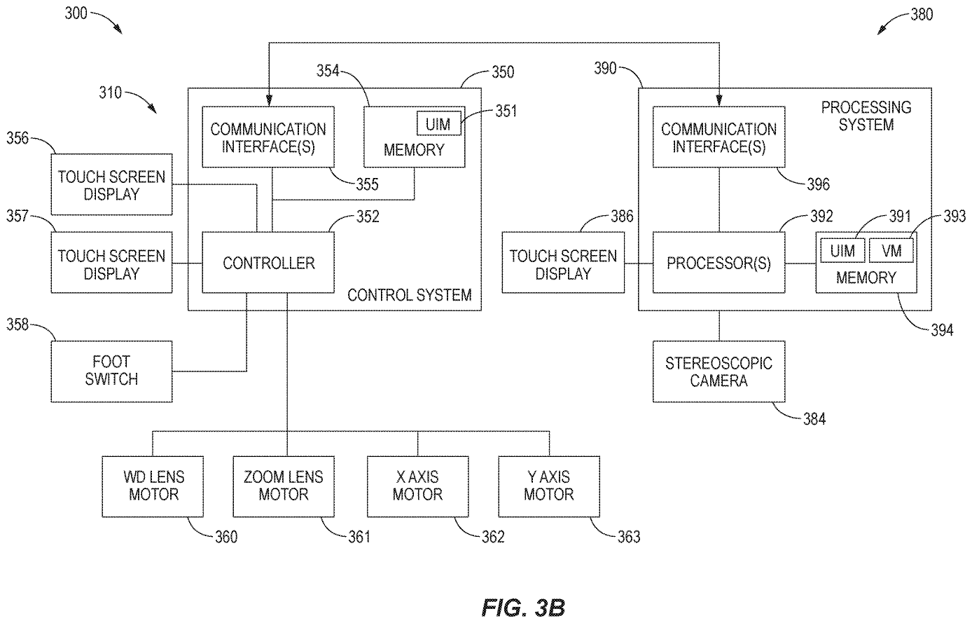

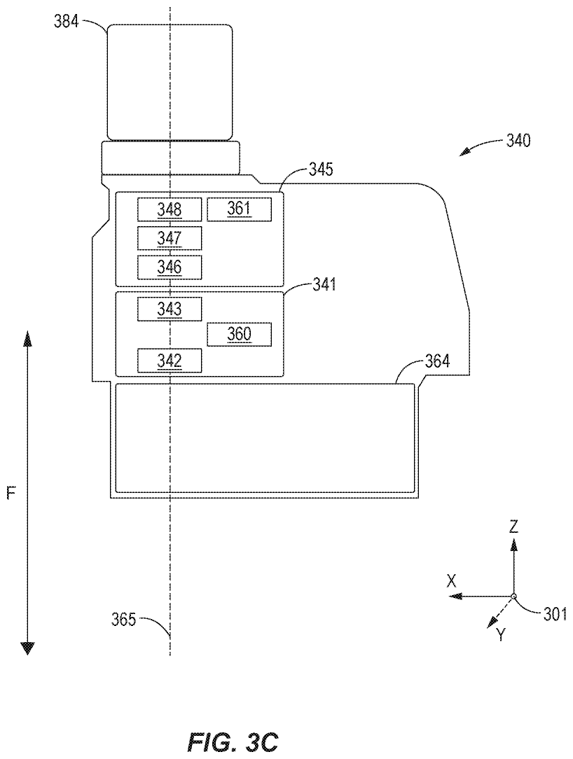

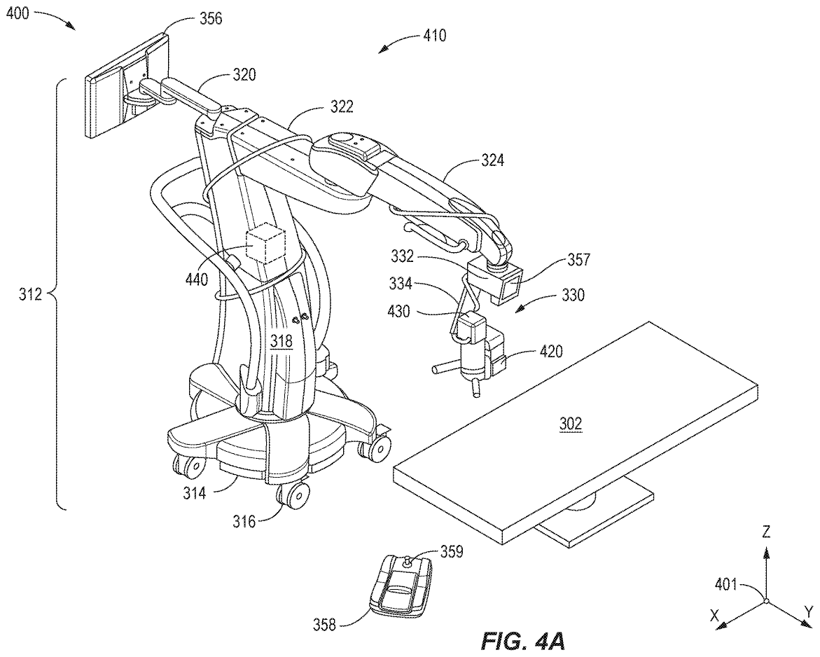

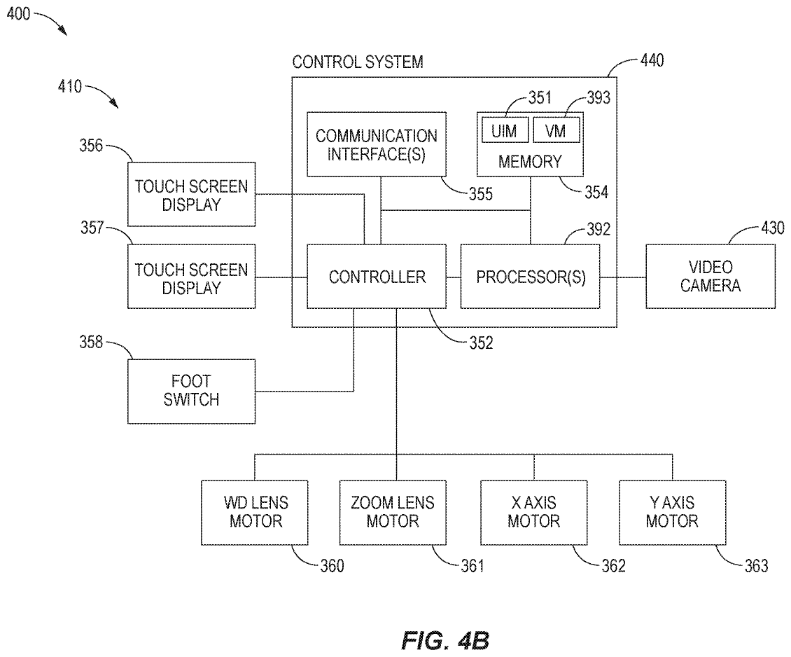

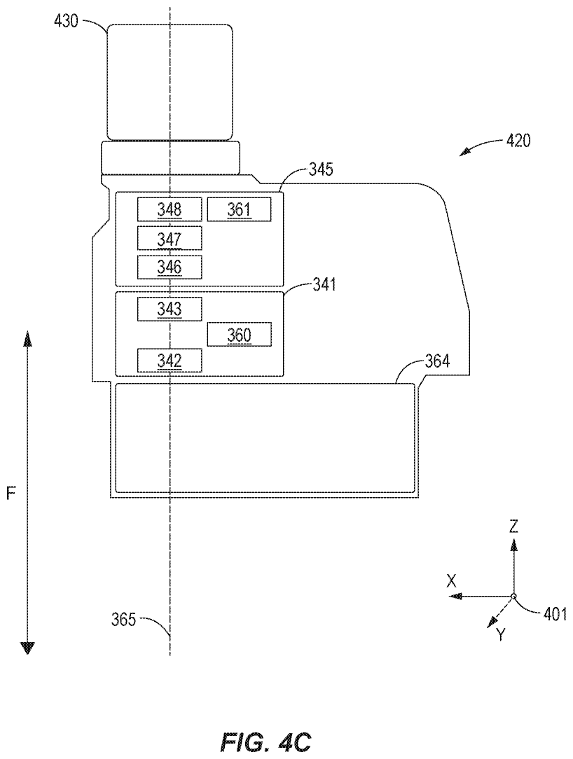

Examples of optical imaging systems are described below with respect to FIGS. 3A, 3B, 3C, 4A, 4B, and 4C.

FIG. 3A depicts an optical imaging system 300, in accordance with certain embodiments of the present disclosure. An orthogonal coordinate system 301 is depicted and includes X, Y, and Z axes.

In certain embodiments, the optical imaging system 300 includes an ophthalmic microscope 310 and an ophthalmic surgical image processing system 380 located proximate to the patient bed 302.

The ophthalmic microscope 310 includes a floor stand 312 and a microscope optical head 330. The floor stand 312 includes, among other things, a support base 314 having a set of wheels 316 with brakes, a support pillar 318 that is rotatably coupled to the support base 314, a support arm 322 that is attached to the support pillar 318, and an articulated arm 324 that is rotatably coupled to the support arm 322. The articulated arm 324 may be raised and lowered to place the microscope optical head 330 at a preferred height over the head of a patient disposed on the patient bed 302.

The microscope optical head 330 includes a base unit 332 that is rotatably coupled to the end of the articulated arm 324, a yoke 334 that is coupled to the base unit, a stereoscopic microscope 340 that is rotatably coupled to the yoke 334, and a stereoscopic camera 384 attached to the stereoscopic microscope 340. The base unit 332 includes an X axis motor and a Y axis motor that are configured to move the stereoscopic microscope 340 in the XY plane. In some embodiments, the base unit 332 also includes a Z axis motor that is configured to move the stereoscopic microscope 340 in the Z direction.

A control system 350 is located within the floor stand 312 (such as within the support pillar 318) and is configured to control the operation of the microscope optical head 330. The control system 350 may control certain functions of the stereoscopic microscope 340 (such as the focus, the magnification, the illumination, the inverter, and so on), the position of the stereoscopic microscope 340 in two- or three-dimensional space (such as orthogonal X, Y directions, orthogonal X, Y, Z directions, and so on).

A touch screen display 356 may be coupled to the control system 350 and attached to the support pillar 318 via a movable arm 320. Another touch screen display 357 may be coupled to the control system 350 and attached to the microscope optical head 330. The control system 350 generates and presents a user interface (UI) on the touch screen display 356 and another UI on the touch screen display 357. The touch screen displays 356 and 357 are configured to receive inputs from the surgeon and provide those inputs to the control system 350 to control certain functions of the stereoscopic microscope 340, the position of the stereoscopic microscope 340 in two- or three-dimensional space, and so on.

A wireless (or wired) foot switch 358 is also coupled to the control system 350 and includes a joystick 359 and control switches that may be actuated by the surgeon. Similar to the UIs, the foot switch 358 receives inputs from the surgeon and provides those inputs to the control system 350 to control certain functions of the stereoscopic microscope 340, the position of the stereoscopic microscope 340 in two- or three-dimensional space, and so on.

The ophthalmic surgical image processing system 380 includes a console 382, the stereoscopic camera 384 attached to the stereoscopic microscope 340, a touch screen display 386 attached to the console 382, a signal cable 388, and a processing system 390. The signal cable 388 couples the stereoscopic camera 384 to the processing system 390. The stereoscopic camera 384 generates a video stream that is provided to the processing system 390 for further processing. The video stream includes a sequence of video frames, and each video frame includes stereoscopic image data (also known as left and right 2D image data). The video stream may have a video frame rate of 30 fps, 60 fps, at least 30 fps, and so on. The processing system 390 may derive range data from the left and right 2D image data to generate three-dimensional stereoscopic image data (also known as 3D image data).

In certain embodiments, the processing system 390 is located within the console 382. In some embodiments, the processing system 390 may be located in the floor stand 312 (e.g., in the support pillar 318), and the console 382 and the signal cable 388 are not provided. The touch screen display 386 may be attached to the floor stand 312, a wall, a ceiling, or a separate floor stand, and may be coupled to the processing system 390 via a signal cable or a wireless communication link. Alternatively, the touch screen display 356 may be configured to replace the touch screen display 386.

The processing system 390 is configured to receive and process the video stream from the stereoscopic camera 384 and then display the processed stereoscopic image data on the touch screen display 386 (and/or the touch screen displays 356, 357). Additionally, the processing system 390 is configured to, among other things, generate an overlay that includes a safe insertion region and one or more trocar insertion locations that lie within the safe insertion region, and present the overlay with the image on the touch screen display 386 (and/or the touch screen displays 356, 357), as described in more detail below. In some embodiments, the overlay does not include the safe insertion region. In some embodiments, the processing system 390 may be configured to present the overlay onto a display that is located in the optical pathway of the stereoscopic microscope 340 for viewing with the image through the binoculars.

The processing system 390 is coupled to the control system 350, via the signal cable 388 or a separate electrical or optical signal cable, and may also be configured to send certain processed data to the controller 352. In some embodiments, the signal cable 388 may be replaced by a wireless communication link between the control system 350 and the processing system 390.

FIG. 3B presents a block diagram of the optical imaging system 300, in accordance with certain embodiments of the present disclosure.

The optical imaging system 300 includes the ophthalmic microscope 310 and the ophthalmic surgical image processing system 380. The ophthalmic microscope 310 includes, among other things, the control system 350, the touch screen displays 356 and 357, the foot switch 358, an X axis motor 362, a Y axis motor 363, a working distance (WD) lens motor 360, and a zoom lens motor 361.

The control system 350 includes, among other things, a controller 352, a memory 354 coupled to the controller 352, and one or more communication interfaces 355 coupled to the controller 352. The controller 352 is coupled to the touch screen displays 356 and 357, the foot switch 358, the X axis motor 362, the Y axis motor 363, the WD lens motor 360, and the zoom lens motor 361.

The controller 352 may be a processor, a microprocessor, a microcontroller, a programmable logic controller (PLC), a field programmable gate array (FPGA), an application specific integrated circuit (ASIC), and so on.

The memory 354 may include any combination of random access memory (RAM), dynamic RAM (DRAM), static RAM (SRAM), read only memory (ROM), flash memory, cache memory, and/or any other type of non-transitory computer-readable medium. The memory 354 stores a user interface module (UIM) 351 that is executed by the controller 352.

The communication interface 355 may include one or more wired or wireless communication interfaces, such as a Universal Serial Bus (USB) interface, an Ethernet interface, a WiFi interface, a Bluetooth interface, and so on.

As described above, the controller 352 is configured to receive inputs from the surgeon, via the UIs presented on the touch screen displays 356 and/or 357 and/or the foot switch 358, to control certain functions of the stereoscopic microscope 340, the position of the stereoscopic microscope 340 in two- or three-dimensional space, and so on. For example, in response to receiving a command from an input device (e.g., the foot switch 358, and so on.), the controller 352 may command the X axis motor 362 and/or the Y axis motor 363 to move the stereoscopic microscope 340 in the XY plane.

The ophthalmic surgical image processing system 380 includes, among other things, the stereoscopic camera 384, the processing system 390, and the touch screen display 386. The processing system 390 includes, among other things, a processor 392, a memory 394 coupled to the processor 392, and one or more communication interfaces 396 coupled to the processor 392.

The processor 392 may be a microprocessor, a microcontroller, a programmable logic controller (PLC), a field programmable gate array (FPGA), an application specific integrated circuit (ASIC), and so on. In certain embodiments, the processor 392 may include one or more single-core or multi-core processors that are configured to process stereoscopic image data, execute ML processes, and so on. In other embodiments, the processor 392 may be a system-on-chip (SoC) that includes one or more single-core or multi-core processors and one or more special processors, such as a graphics processing unit (GPU), a neural processing unit (NPU), and so on, that are configured to process stereoscopic image data, execute ML processes, and so on.

Generally, the processor 392 is configured to receive and process the video stream from the stereoscopic camera 384 via the signal cable 388 and then present the processed stereoscopic image data on the touch screen display 386. Additionally, the processor 392 is configured to identify the limbus of the eye, determine a safe insertion region, determine one or more trocar insertion locations within the safe insertion region, generate an overlay including the safe insertion region and the trocar insertion locations, and present the overlay with the image on the touch screen display 386 (and/or the touch screen displays 356, 357), as described in more detail below. In some embodiments, the overlay does not include the safe insertion region. The processor 392 may also be configured to send certain processed data to the controller 352.

The memory 394 may include any combination of random access memory (RAM), dynamic RAM (DRAM), static RAM (SRAM), read only memory (ROM), flash memory, cache memory, and/or any other type of non-transitory computer-readable medium. The memory 394 stores a user interface module (UIM) 391 and a visualization module (VM) 393 that are executable by the processor 392. The VM 393 may be configured to process stereoscopic image data (e.g., left and right images).

The communication interface 396 may include one or more wired or wireless communication interfaces, such as a Universal Serial Bus (USB) interface, an Ethernet interface, a WiFi interface, a Bluetooth interface, and so on. The communication interface 396 is coupled to the communication interface 355 using a wired (or wireless) connection, and the processor 392 exchanges data with the controller 352 over the wired (or wireless) connection.

FIG. 3C depicts a left side view of the stereoscopic microscope 340, in accordance with certain embodiments of the present disclosure. The orthogonal coordinate system 301 is also depicted.

Generally, the stereoscopic microscope 340 includes an illumination system 364, an optical assembly 341, a zoom assembly 345, and left and right optical units, such as binoculars with left and right lenses, prisms, and eyepieces. In certain embodiments, the stereoscopic camera 384 may replace the binoculars, while in other embodiments, the stereoscopic camera 384 may be used in combination with the binoculars. The stereoscopic camera 384 provides left and right optical sensors. The stereoscopic microscope 340 has an optical axis 365.

The optical assembly 341 includes a front working distance lens 342 and a rear working distance lens 343 disposed within a housing. A focal plane is located at a distance equal to the focal length F from a principal plane of the optical assembly 341. The optical assembly 341 is configured to provide a variable working distance W, which sets a plane of the target site or scene that is in focus. The controller 352 (not shown in FIG. 3C) is configured to selectively command the WD lens motor 360 coupled to the rear working distance lens 343 (and/or the front working distance lens 342) to change the spacing between the rear working distance lens 343 and the front working distance lens 342 to adjust the focus in response to input from the surgeon, such as a focus position received from the foot switch 358 or from the UI displayed on the touch screen display 357. The optical assembly 341 is configured to provide a single view of the target site to the stereoscopic camera 384 via the left optical path or the right optical path.

The zoom assembly 345 includes zoom lenses that change a focal length and angle of view of the target site to provide zoom magnification. In certain embodiments, the zoom assembly 345 includes a front lens set 346, a zoom lens unit 347, and a lens barrel set 348. The front lens set 346 may include respective positive converging lenses (for the right and left optical paths) to direct light to respective lenses in the zoom lens unit 347. The lateral position of the front lens set 346 may define a beam from the optical assembly 341 that is propagated to the zoom lens unit 347. The front lens set 346 may include lenses with adjustable radial and axial positions.

The zoom lens unit 347 may form an afocal zoom system for changing the size of a field-of-view (e.g., a linear field-of-view) by changing the size of the light beam propagated to the lens barrel set. The zoom lens unit 347 may include a front zoom lens set with respective right and left front zoom lenses. The zoom lens unit 347 may include a rear zoom lens set with respective right and left front zoom lenses. The front zoom lens set may include positive converging lenses while the rear zoom lens set may include negative diverging lenses. The controller 352 is configured to selectively command the zoom lens motor 361 to move the lens barrel set 348 along the optical axis 365 to increase or decrease the magnification in response to input from the surgeon, such as a magnification position received from the foot switch 358 or from the UI displayed on the touch screen display 357.

FIG. 4A depicts an optical imaging system 400, in accordance with certain embodiments of the present disclosure. An orthogonal coordinate system 401 is depicted and includes X, Y, and Z axes.

In certain embodiments, the optical imaging system 400 includes an ophthalmic microscope 410, which includes many of the same components as the ophthalmic microscope 310 (which are identified using the same reference characters). Notably, the microscope optical head 330 includes a monoscopic microscope 420 that is rotatably coupled to the yoke 334, and a video camera 430 that is attached to the monoscopic microscope 420. The video camera 430 generates a video stream that is provided to the control system 440 for further processing. The video stream includes a sequence of video frames, and each video frame includes monoscopic (or 2D) image data. The video stream may have a video frame rate of 30 fps, 60 fps, at least 30 fps, and so on.

The control system 440 is located within the floor stand 312 (such as within the support pillar 318) and is configured to control the operation of the microscope optical head 330. The control system 440 may control certain functions of the monoscopic microscope 420 (such as the focus, the magnification, the illumination, the inverter, and so on), the position of the monoscopic microscope 420 in two- or three-dimensional space (such as orthogonal X, Y directions, orthogonal X, Y, Z directions, and so on).

The control system 440 is configured to receive and process the video stream from the video camera 430 and then display the processed monoscopic image data on the touch screen display 356 (and/or the touch screen display 357). Additionally, the control system 440 is configured to, among other things, generate an overlay that includes a safe insertion region and one or more trocar insertion locations that lie within the safe insertion region, and present the overlay with the image on the touch screen display 356 (and/or touch screen display 357), as described in more detail below. In some embodiments, the overlay does not include the safe insertion region. In some embodiments, the control system 440 may be configured to present the overlay onto a display that is located within the optical pathway of the monoscopic microscope 420 for viewing with the image through the eyepieces.

FIG. 4B presents a block diagram of the optical imaging system 400, in accordance with certain embodiments of the present disclosure.

In certain embodiments, the optical imaging system 400 includes an ophthalmic microscope 410. The ophthalmic microscope 410 is generally the same as the ophthalmic microscope 310, and the control system 440 is generally the same as the control system 350. The common components between the ophthalmic microscope 410 and the ophthalmic microscope 310 are identified in FIG. 4B using the same reference numbers used in FIG. 3B.

The control system 440 includes the processor 392 and the VM 393 from the processing system 390 depicted in FIG. 3B. The VM 393 is stored in the memory 354 and may be configured to process monoscopic image data (e.g., single images) instead of (or in addition to) stereoscopic image data.

Generally, the processor 392 is configured to receive and process monoscopic image data from the video camera 430 and then present the processed monoscopic image data on the touch screen display 356 (and/or touch screen display 357). Additionally, the processor 392 is configured to identify the limbus of the eye, determine a safe insertion region, determine one or more trocar insertion locations within the safe insertion region, generate an overlay including the safe insertion region and the trocar insertion locations, and present the overlay with the image on the touch screen display 356 (and/or touch screen display 357), as described in more detail below. In some embodiments, the overlay does not include the safe insertion region. The processor 392 may also be configured to send certain processed data to the controller 352.

FIG. 4C depicts a left side view of the monoscopic microscope 420, in accordance with certain embodiments of the present disclosure. The orthogonal coordinate system 401 is also depicted.

Generally, the monoscopic microscope 420 includes an illumination system 364, a monoscopic version of the optical assembly 341, a monoscopic version of the zoom assembly 345, an optical unit, prisms, and eyepieces. The monoscopic version of the optical assembly 341 and the monoscopic version of the zoom assembly 345 function as described above using a single optical path (e.g., the left optical path). In certain embodiments, the video camera 430 replaces the eyepieces and provides an optical sensor, while in other embodiments, the video camera 430 may be used in combination with the eyepieces. The monoscopic microscope 420 has an optical axis 365.

As described above, embodiments of the present disclosure advantageously provide systems and methods that provide integrated, precise, real-time visualization guidance for trocar placement during ophthalmic surgical procedures, such as vitreoretinal procedures. The visualization guidance does not rely on measuring instruments and surgical marking instruments, which eliminates the variability associated with manual measurement techniques, provides consistent, precise guidance across different patients and surgical procedures, reduces the time required to determine insertion locations during ophthalmic surgery, and enhances surgical safety.

More particularly, the optical imaging systems 300, 400 provide real-time, ML-based techniques that identify anatomical landmarks of the eye (such as the limbus) from a surgical microscope video stream, determine a safe insertion region and one or more trocar insertion locations, and provide visualization guidance as an overlay to the video stream that is presented on a display of the optical imaging systems 300, 400 during the ophthalmic surgery.

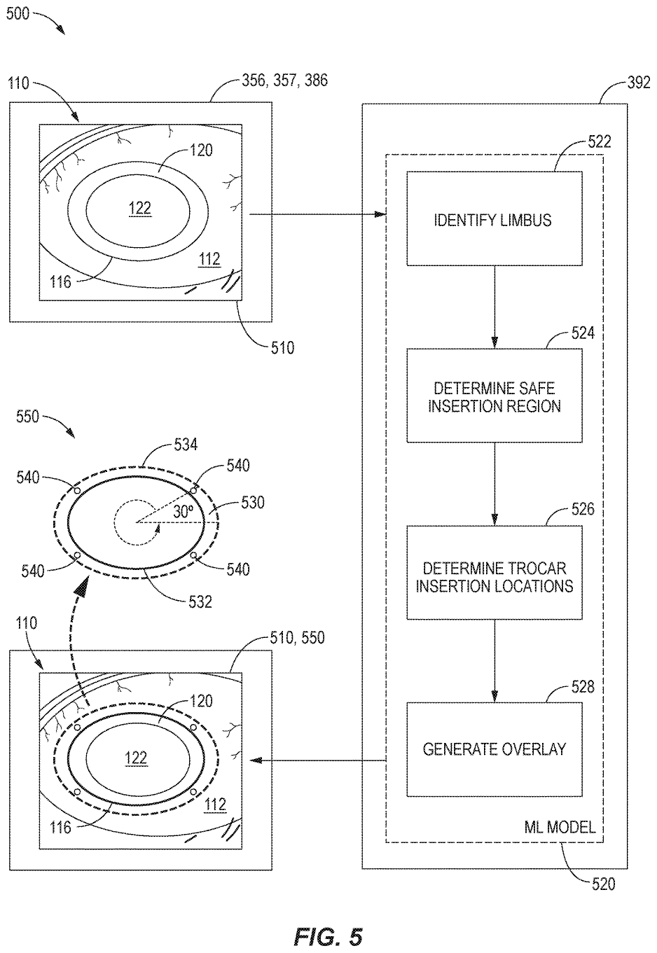

FIG. 5 depicts a data flow diagram of a method 500 for providing visualization guidance for trocar placement during ophthalmic surgical procedures, in accordance with embodiments of the present disclosure. The method 500 is performed by an ophthalmic imaging system, such as the optical imaging systems 300, 400 described above in relation to FIGS. 3A-3C and 4A-4C (respectively).

For example, as described above with respect to FIGS. 3A-3C, the optical imaging system 300 includes an ophthalmic microscope 310 and an ophthalmic surgical image processing system 380. The stereoscopic camera 384 is attached to the stereoscopic microscope 340 and generates a video stream that is provided to the ophthalmic surgical image processing system 380 for further processing. The video stream includes a sequence of video frames, and each video frame includes stereoscopic image data of the eye during the ophthalmic surgery.

Generally, the processor 392 of the ophthalmic surgical image processing system 380 processes the stereoscopic image data in real time to identify anatomical features of the eye and determine trocar placement guidance that is presented to the surgeon as an overlay to the video stream that is presented on the touch screen display 386 (and/or the touch screen displays 356, 357). In some embodiments, the overlay may be projected onto a display within the stereoscopic microscope 340 which is visible through the binoculars. As discussed herein, “real-time” processing refers to the processing of image data and the determination of trocar insertion locations with sufficiently low latency that the overlay is generated and available substantially simultaneously with the input image data in each video frame, enabling immediate use without perceptible delay to the surgeon.

With respect to FIG. 5, the processor 392 receives the video stream from the stereoscopic camera 384. Each video frame comprises image data 510 of the eye 110 of the patient. The sclera 112, the limbus 116, the iris 120, and the pupil 122 of the eye 110 are identified. In this embodiment, the image data 510 may be left 2D image data, right 2D image data, or 3D image data. In some embodiments, the processor 392 may preprocess the image data 510 to normalize illumination conditions and enhance contrast prior to identifying the limbus 116.

For another example, as described above with respect to FIGS. 4A-4C, the optical imaging system 400 includes an ophthalmic microscope 410. The video camera 430 is attached to the monoscopic microscope 420 and generates a video stream that is provided to the control system 440 for further processing. The video stream includes a sequence of video frames, and each video frame includes monoscopic image data of the eye during the ophthalmic surgery. Generally, the processor 392 of the control system 440 processes the monoscopic image data in real time to identify anatomical features of the eye and determine trocar placement guidance that is presented to the surgeon as an overlay to the video stream that is presented on the touch screen displays 356, 357 and/or 386. In some embodiments, the overlay may be projected onto a display within the monoscopic microscope 420 which is visible through the eyepieces.

With respect to FIG. 5, the processor 392 receives the video stream from the video camera 430. Each video frame comprises image data 510 of the eye 110 of the patient. In this embodiment, the image data 510 is monoscopic (or 2D) image data. In some embodiments, the processor 392 may preprocess the image data 510 to normalize illumination conditions and enhance contrast prior to identifying the limbus 116.

The functionality described at blocks 522, 524, 526, and 528 is performed by the processor 392 of the processing system 390 or the processor 392 of the control system 440. In certain embodiments, the processor 392 may execute an ML model 520 to perform some or all of the functionality described at blocks 522, 524, 526, and 528. The ML model 520 may comprise one or more models, such as a single model, a group of models, and so on. For example, although each of the blocks 522, 524, 526, and 528 is described as being performed by the ML model 520, each block could be performed by the same model, a different model in a group of models, and so on, referred to herein as the ML model 520.

At block 522, the ML model 520 processes the image data 510 to identify the limbus 116 of the eye 110. In certain embodiments, the ML model 520 comprises a deep learning segmentation model that includes one or more neural networks. The ML model 520 is configured to generate a segmentation mask, such as a binary segmentation mask, that identifies the limbus 116 based on the image data 510. In other words, the ML model 520 is configured to extract a limbus contour from the segmentation mask. The segmentation mask distinguishes the limbus 116 from other anatomical structures in the image data 510, such as the pupil 122, the inner boundary of the iris 120, and so on. In various embodiments, the segmentation mask may include a binary mask, a multi-class mask, a probability map, a confidence-weighted mask, or other pixel-wise representations identifying limbus likelihood.

In certain embodiments, the ML model 520 may be trained using a supervised learning approach based on labeled ophthalmic image data. For example, a convolutional neural network (CNN) having a U-Net architecture may be trained on a dataset of surgical or diagnostic images of eyes in which the limbus has been annotated, such as through pixel-level segmentation masks identifying the limbus boundary. During training, each input image is provided to the model along with a corresponding ground truth mask, and the model learns to generate a predicted segmentation mask that distinguishes the limbus from surrounding anatomical structures.

The training process may include optimizing model parameters using a loss function that quantifies differences between the predicted masks and the ground truth masks, such as a cross-entropy loss, Dice loss, or a combination thereof. The dataset may include images captured under varying lighting conditions, magnifications, and anatomical variations to improve generalization. Through iterative training, the model learns spatial and textural features associated with the limbus, enabling the trained model to identify and segment the limbus in new image data acquired during an ophthalmic surgical procedure.

Based on the segmentation mask, the ML model 520 determines geometric characteristics of the limbus 116. For example, the ML model 520 may determine a contour corresponding to the limbus 116 and perform a curve fitting operation to determine a center location and a radius of the limbus 116. The ML model 520 uses the geometric characteristics to define a spatial relationship between the limbus 116 and other regions of the eye 110.

In certain embodiments, the ML model 520 may be trained to determine geometric characteristics of the limbus based on the segmentation mask generated for the limbus. For example, the ML model 520 may be trained using supervised learning on a dataset that includes segmentation masks of the limbus along with corresponding ground truth geometric parameters, such as a center location and a radius of the limbus. The ML model 520 may receive, as input, the segmentation mask (or features derived from the segmentation mask) and may be trained to regress the geometric parameters associated with the limbus. In some embodiments, the ML model 520 may comprise a neural network configured to learn relationships between the spatial distribution of the segmented limbus pixels and the corresponding geometric characteristics.

The training process may involve optimizing a loss function that penalizes deviations between predicted geometric parameters and ground truth values, such as mean squared error. In other embodiments, the ML model 520 may be configured to refine or complement geometric computations (e.g., circle or ellipse fitting) by learning to correct for distortions, noise, or partial occlusions present in the segmentation mask. Through training on diverse datasets that include variations in eye orientation, image quality, and anatomical differences, the model may learn to robustly determine geometric characteristics of the limbus from segmentation masks generated during an ophthalmic surgical procedure.

At block 524, the ML model 520 determines, based at least in part on the geometric characteristics of the limbus 116, a safe insertion region 530 located posterior to the limbus 116 for trocar insertion. In certain embodiments, the safe insertion region 530 corresponds to a pars plana region of the eye 110. The determination of the safe insertion region 530 may include calculating a position of the safe insertion region 530 based on a geometric relationship between a radius of the limbus 116 and a radius of the eye 110.

The safe insertion region 530 includes an upper boundary 532 and a lower boundary 534. The upper boundary 532 of the safe insertion region 530 is the contour of the limbus 116 determined in block 522. The lower boundary 534 is located at a predetermined distance posterior to the upper boundary 532, such as 3.5-4 mm posterior to the upper boundary 532 for phakic eyes, 3-3.5 mm posterior to the upper boundary 532 for pseudophakic eyes, ~1.5 mm posterior to the upper boundary 532 for infant eyes, and so on. In some embodiments, the safe insertion region 530 may be defined using one or more anatomical, geometric, statistical, or learned constraints, and need not be limited to a fixed-width band.

In certain embodiments, the ML model 520 may be trained to determine a safe insertion region located posterior to the limbus based at least in part on geometric characteristics of the limbus. For example, the ML model 520 may be trained using supervised learning on a dataset that includes geometric representations of the limbus (e.g., center location, radius, and orientation) along with corresponding ground truth labels defining safe insertion regions for trocar placement. These ground truth labels may be derived from clinical guidelines, expert annotations, or patient-specific anatomical data. The model may receive, as input, the geometric characteristics of the limbus and, in some embodiments, additional parameters such as estimated eye radius or biometric measurements, and may be trained to output parameters defining the safe insertion region, such as a radial offset, angular position, or boundary representation. The training process may involve optimizing a loss function that measures differences between predicted safe regions and ground truth regions, enabling the model to learn relationships between limbus geometry and anatomically appropriate insertion zones. Through training on diverse datasets that capture variations in eye anatomy and imaging conditions, the model may learn to determine safe insertion regions that are consistent with anatomical constraints and surgical best practices.

In some embodiments, the processor 392 may receive patient-specific biometric data associated with the eye 110, and the ML model 520 may determine the safe insertion region 530 based on the patient-specific biometric data as well as the geometric characteristics of the limbus 116. The patient-specific biometric data may include ocular measurements obtained from a preoperative diagnostic device or other data sources. These patient-specific biometric data are used to improve the accuracy of anatomical calculations performed by the ML model 520.

At block 526, the ML model 520 determines one or more trocar insertion locations 540 within the safe insertion region 530. In certain embodiments, the trocar insertion locations 540 are arranged at different angular positions within the safe insertion region 530. For example, the trocar insertion locations 540 may correspond to a four-trocar configuration (illustrated in FIG. 5) in which four trocar insertion locations 540 are arranged at 30°, 150°, 210°, and 330° relative to a horizontal reference line extending through the center of the safe insertion region 530.

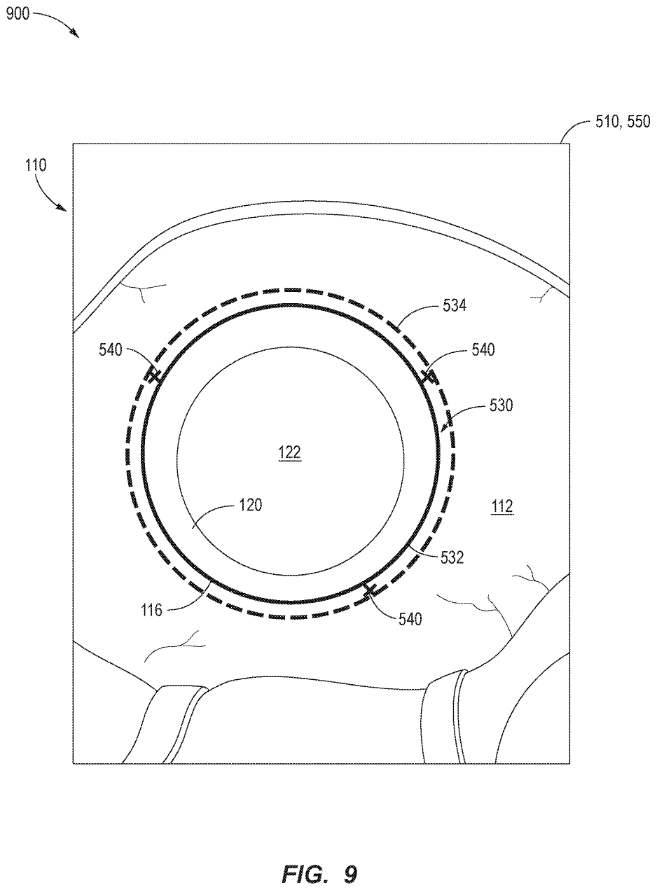

In certain embodiments, the determination of trocar insertion locations 540 may consider various conditions of the patient's eye, in addition to the 3D eye biometry, and leverage that information to provide locations that are not available in the known manual measurement process using fixed-distance rulers. The locations may be clinically important to setup the stage for successful vitreoretinal surgery, especially for trainees who are not fully aware of or considering the various conditions in the patient's eye. For example, these conditions may include whether the patient's eye is Phakic (natural crystalline lens) or Pseudophakic (implanted with IOL), whether there is a pathological pattern in the patient's eye (e.g., where a retina detachment is located), the surgeon's treatment plan, placing the trocars to ensure that the instrument with right length can reach the treatment area (e.g., macular peeling), and so on. Other trocar configurations are also supported, such as a single trocar configuration, a two-trocar configuration, a three-trocar configuration (illustrated in FIG. 9), and so on. Additionally or alternatively, the locations may be based on avoiding certain anatomical features, such as heavily vascularized regions of the sclera, or other anatomical aspects of an eye generally or of a given patient specifically.

In certain embodiments, the ML model 520 may be trained to determine one or more trocar insertion locations within a previously determined safe insertion region based on supervised learning techniques. For example, the ML model 520 may be trained using a dataset that includes representations of safe insertion regions, along with ground truth labels identifying preferred trocar insertion locations within those regions. These labels may be derived from expert annotations, historical surgical data, or predefined surgical configurations (e.g., three-trocar or four-trocar arrangements at specific angular positions). The model may receive, as input, a representation of the safe insertion region, and in some embodiments additional contextual information such as limbus geometry, eye orientation, or patient-specific parameters, and may be trained to output one or more insertion locations defined by spatial coordinates or angular positions.

The training process may involve optimizing a loss function that penalizes deviations between predicted insertion locations and ground truth locations, enabling the model to learn placement patterns that satisfy anatomical constraints and surgical preferences. Through training on datasets that capture variations in anatomy, surgical approach, and configuration selection, the model may learn to determine trocar insertion locations that are appropriately positioned within the safe insertion region for a given surgical procedure.

At block 528, the ML model 520 generates an overlay 550 that includes the safe insertion region 530 and the one or more trocar insertion locations 540. The safe insertion region 530 is indicated by a graphical element representing the upper boundary 532 (such as a contour line) and a graphical element representing the lower boundary 534 (such as a boundary line). The trocar insertion locations 540 are indicated by another type of graphical element (such as a circle, a cross, and so on).

The processor 392 then presents the overlay 550 with the image data 510 on a display of the optical imaging system, such as the touch screen display 386 (and/or the touch screen displays 356, 357) of the optical imaging system 300 or the touch screen displays 356, 357 of the optical imaging system 400. In some embodiments, the processor 392 presents the overlay 550 with the image data 510 on a display within the stereoscopic microscope 340 which is visible through the binoculars, or on a display within the monoscopic microscope 420 which is visible through the eyepieces.

The safe insertion region 530 and the trocar insertion locations 540 are determined in real-time based on the image data 510 within each video frame of the video stream. Accordingly, as the eye 110 moves during the surgical procedure, the safe insertion region 530 and the trocar insertion locations 540 follow the movement of the eye 110 in the video stream. Advantageously, the eye 110 does not need to be centered in each video frame in order to calculate the safe insertion region 530 and the trocar insertion locations 540 in each video frame.

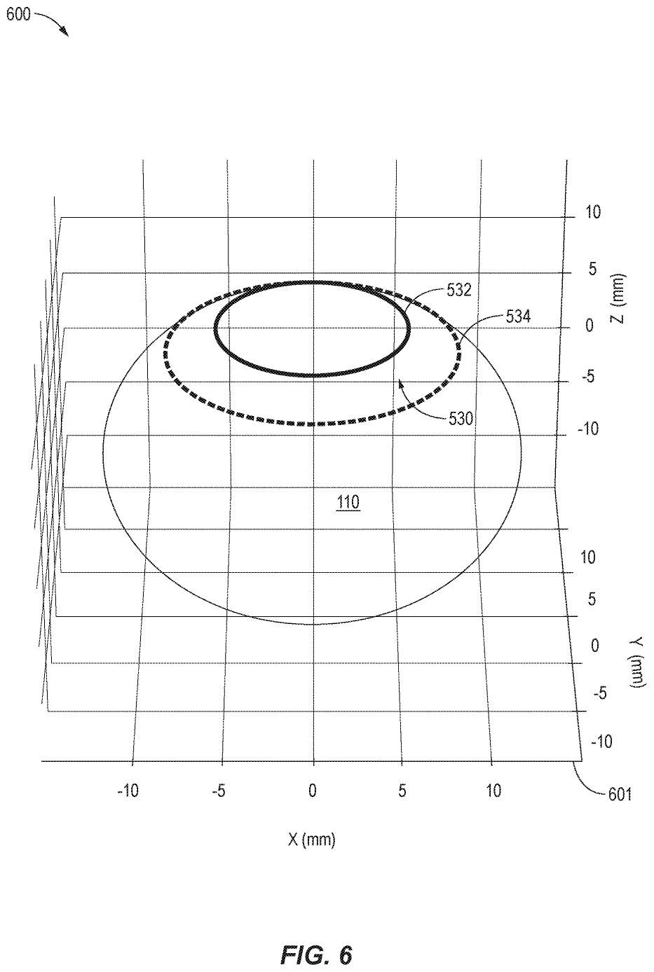

FIG. 6 depicts a 3D view 600 of an eye 110, in accordance with an embodiment of the present disclosure.

The 3D view 600 includes an XYZ coordinate system 601 that includes an X axis (distance in mm), a Y axis (distance in mm), and a Z axis (distance in mm). The center of the eye 110 is located at the coordinates 0,0,0, and the diameter of the eye 110 is about 24 mm.

The safe insertion region 530, the upper boundary 532, and the lower boundary 534 are depicted.

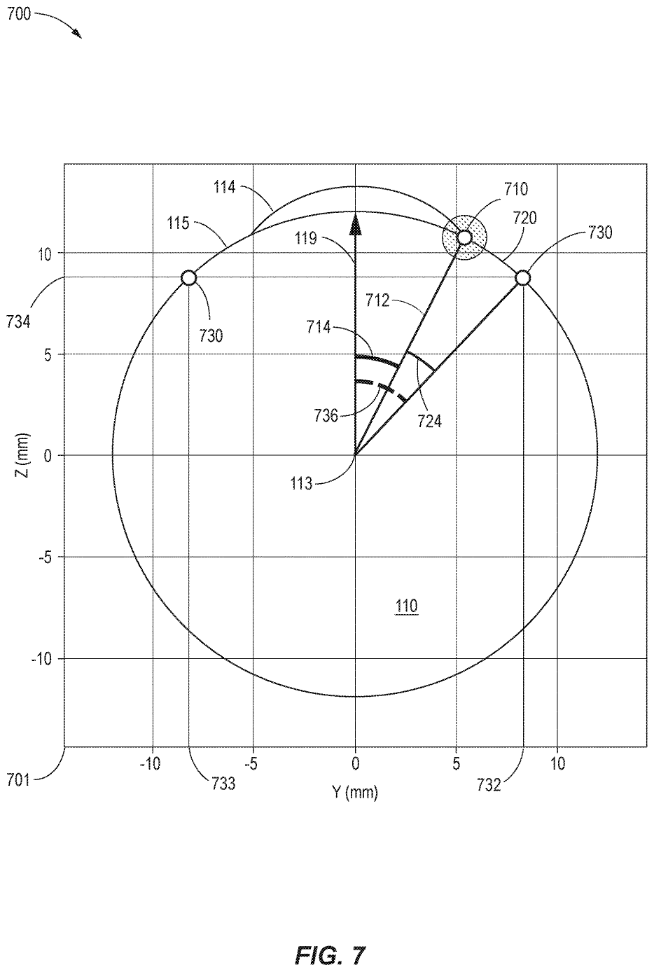

FIG. 7 depicts a 2D cross-sectional view 700 of the eye 110 in the YZ plane, in accordance with embodiments of the present disclosure.

The 2D cross-sectional view 700 includes a YZ coordinate system 701 that includes the Y axis (distance in mm) and the Z axis (distance in mm). At the center of the eye 110 (labeled as the eye center 113) are the coordinates 0,0, and the diameter of the eye 110 is about 24 mm. The circular outline 115 of the eye 110 and the cornea 114 in the YZ plane are also depicted.

As discussed above with respect to FIG. 5, the ML model 520 processes the image data 510 to identify the limbus 710 of the eye 110 at block 522. In certain embodiments, the position of the limbus 710 in three-dimensional space is determined based on geometric relationships associated with the eye 110. The limbus 710 is identified from image data 510, and a radius of the limbus 710 (labeled as the limbus radius 712) may be determined from a segmentation mask of the limbus 710. The limbus radius 712 may be expressed in physical units by converting pixel-based measurements obtained from the segmentation mask.

The limbus 710 corresponds to the upper boundary 532 of the safe insertion region 530 depicted in FIG. 5.

A limbus angle 714 is then determined based on the relationship between the limbus radius 712 and the radius of the eye 110 (labeled as the eye radius 119). The limbus angle 714 represents an angular position of the limbus 710 relative to the eye center 113 in three-dimensional space (Equation 1).

$${θ}_{1} = arcsin ( \frac{{r}_{limbus}}{{R}_{eye}} ) \qquad Eq . 1$$

where r_{limbus} represents the limbus radius 712 and R_{eye} represents the eye radius 119.

In certain embodiments, a target insertion distance posterior to the limbus 710 is defined along a curved surface 720 of the eye 110. For example, an insertion distance of approximately 3.5 mm may be used for certain surgical procedures. This distance may be converted into an angular offset 724 relative to the eye radius 19 (Equation 2).

$$θ = \frac{d}{{R}_{eye}} \qquad Eq . 2$$

where d represents the insertion distance (e.g., 3.5 mm). For a representative eye radius 119 of approximately 12 mm, the angular offset 724 may be approximately 0.292 radians (approximately 16.7 degrees).

The lower boundary 730 of the safe insertion region is then determined based on the limbus angle 714 and the angular offset 724. In particular, a safe zone angle 736 may be defined as a sum of the limbus 714 and the angular offset 724 (Equation 3).

θ₂=θ₁+θ Eq. 3

Based on the safe zone angle 736, a corresponding Y position 732 of the safe insertion region (Y_{safe}) may be determined (Equation 4).

Y_{safe}=R_{eye}·sin(θ₂) Eq. 4

Similarly, a corresponding Z position 734 of the lower boundary 730 of the safe insertion region (Z_{safe}) may be determined (Equation 5).

Z_{safe}=R_{eye}·cos(θ₂) Eq. 5

The Y position 732, Z position 734 corresponds to a point along a circumferential path defining the lower boundary 730 of the safe insertion region. The lower boundary 730 corresponds to the lower boundary 534 of the safe insertion region 530 depicted in FIG. 5. The Y position 733, Z position 734 corresponds to another point along the circumferential path defining the lower boundary 730 of the safe insertion region.

Accordingly, the safe insertion region 530 is determined in three-dimensional space relative to the limbus and the geometry of the eye. This safe insertion region 530 may then be projected onto the image plane of the image data 510 for visualization, an example of which is illustrated in FIG. 9.

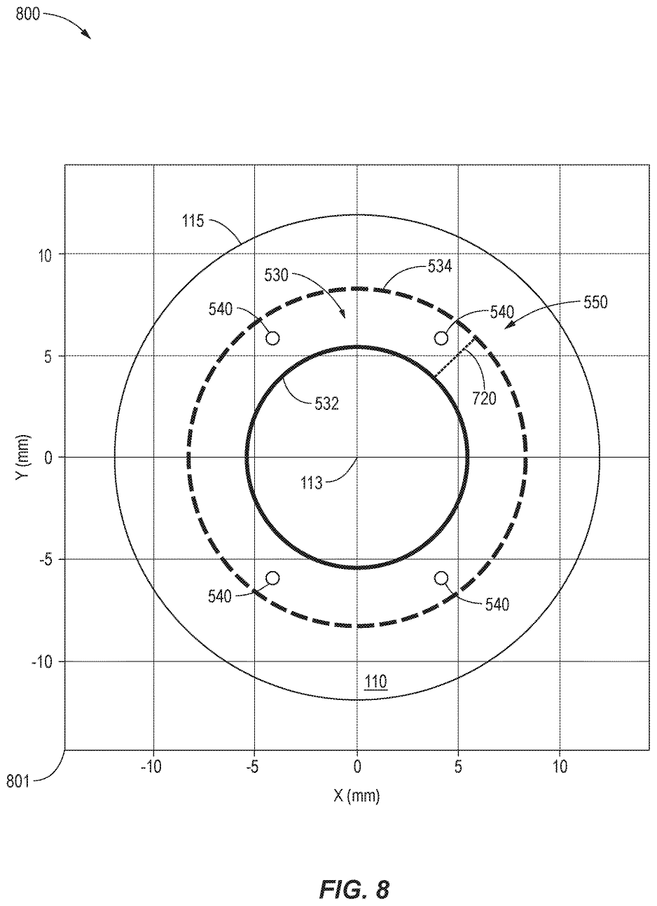

FIG. 8 depicts a 2D projection 800 of the overlay 550 and the circular outline 115 of the eye 110 onto the XY plane, in accordance with embodiments of the present disclosure.

The 2D projection 800 includes an XY coordinate system 801 that includes an X axis (distance in mm) and a Y axis (distance in mm). The eye center 113 is located at the coordinates 0,0, and the diameter of the eye 110 is about 24 mm.

The safe insertion region 530, the upper boundary 532, the lower boundary 534, the trocar insertion locations 540, and the curved surface 720 are depicted.

FIG. 9 depicts a three-trocar configuration, in accordance with embodiments of the present disclosure.

The image data 510 and the overlay 550 are depicted. Additionally, the eye 110, the sclera 112, the limbus 116, the iris 120, the pupil 122, the safe insertion region 530, the upper boundary 532, the lower boundary 534, and the three (3) trocar insertion locations 540 are depicted.

Advantageously, because the overlay 550 is generated for each video frame based on the image data 510 in that video frame, sight rotation or movement of the eye 110, other changes in the eye 110, or a small repositioning of the stereoscopic microscope 340 or the monoscopic microscope 420 from video frame to video frame does not affect the alignment of the overlay 550 with the anatomical features of the eye 110. In other words, movement of the eye 110 from one video frame to the next video frame causes the overlay 550 to identify different locations on the touch screen display 356, 357, and/or 386 for the safe insertion region 530 and the trocar insertion locations 540 in the next video frame.

In certain embodiments, after the trocar insertion locations 540 have been initially determined, the anatomical features of the eye 110 may be tracked in each subsequent video frame and the trocar insertion locations 540 may be updated based on this tracking to ensure that the trocar insertion locations 540 remain at the initially-determined radial positions and circumferential locations. For example, the geometric characteristics of the limbus may be tracked in each video frame, and a spatial transform may be generated based on the geometric characteristics of the limbus in the preceding video frame and the geometric characteristics of the limbus in the current video frame. The spatial transform may then be applied to the trocar insertion locations 540 in the current video frame to ensure that the trocar insertion locations 540 remain at the initially-determined radial positions and circumferential locations.

In some embodiments, the geometric characteristics of the limbus in the preceding video frame may be used in one or more subsequent video frames without generating the segmentation mask and determining the geometric characteristics of the limbus in the subsequent video frame(s). This simplification reduces the processing time to generate the overlay 550 in the subsequent video frame(s).

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. An optical imaging system, comprising:
an ophthalmic microscope, comprising:
a microscope optical head; and
a stereoscopic microscope rotatably coupled to the microscope optical head;
an ophthalmic surgical image processing system comprising:
a stereoscopic camera attached to the stereoscopic microscope, the stereoscopic camera configured to generate a video stream comprising a sequence of video frames, each video frame comprising image data of an eye of a patient;
a display; and
a processing system coupled to the stereoscopic camera and the display, the processing system including a processor configured to:
receive, from the stereoscopic camera, the video stream;
for each video frame in the sequence of video frames:
generate, based on the image data, a segmentation mask identifying a limbus of the eye;
determine, based on the segmentation mask, geometric characteristics of the limbus;
determine, based at least in part on the geometric characteristics of the limbus, a safe insertion region located posterior to the limbus;
determine one or more trocar insertion locations within the safe insertion region;
generate an overlay identifying the one or more trocar insertion locations; and
present, on the display, the overlay with the image data.

2. The optical imaging system of claim 1, wherein movement of the eye from one video frame to the next video frame causes the overlay to identify different locations on the display for the one or more trocar insertion locations in the next video frame.

3. The optical imaging system of claim 1, wherein the overlay further identifies the safe insertion region.

4. The optical imaging system of claim 1, wherein:
the segmentation mask is a binary segmentation mask; and
the processor is configured to determine the geometric characteristics of the limbus by extracting a limbus contour from the binary segmentation mask and performing a curve fitting operation to determine a center location and a radius of the limbus.

5. The optical imaging system of claim 1, wherein the processor is further configured to preprocess the image data to normalize illumination conditions and enhance contrast prior to identifying the limbus.

6. The optical imaging system of claim 1, wherein the processor is further configured to:
receive patient-specific biometric data associated with the eye; and
determine the safe insertion region based on the patient-specific biometric data.

7. The optical imaging system of claim 6, wherein the patient-specific biometric data comprises one or more ocular measurements obtained from a preoperative diagnostic device.

8. The optical imaging system of claim 1, wherein the processor is further configured to determine the safe insertion region by calculating a position of the safe insertion region based on a geometric relationship between a radius of the limbus and a radius of the eye.

9. The optical imaging system of claim 1, wherein the one or more trocar insertion locations include at least three trocar insertion locations arranged at different angular positions within the safe insertion region.

10. The optical imaging system of claim 1, wherein the video stream has a video frame rate of at least 30 frames per second (fps).

11. A method for guiding trocar placement during an ophthalmic surgical procedure, the method comprising:
receiving, from a camera attached to an ophthalmic microscope, a video stream comprising a sequence of video frames, each video frame comprising image data of an eye of a patient;
for each video frame in the sequence of video frames:
generating, based on the image data, a segmentation mask identifying a limbus of the eye;
determining, based on the segmentation mask, geometric characteristics of the limbus;
determining, based at least in part on the geometric characteristics of the limbus, a safe insertion region located posterior to the limbus;
determining one or more trocar insertion locations within the safe insertion region;
generating an overlay identifying the one or more trocar insertion locations; and
presenting, on a display, the overlay with the image data.

12. The method of claim 11, wherein movement of the eye from one video frame to the next video frame causes the overlay to identify different locations on the display for the safe insertion region and the one or more trocar insertion locations in the next video frame.

13. The method of claim 11, wherein the overlay further identifies the safe insertion region.

14. The method of claim 11, wherein:
the segmentation mask is a binary segmentation mask; and
determining the geometric characteristics of the limbus comprises extracting a limbus contour from the binary segmentation mask and performing a curve fitting operation to determine a center location and a radius of the limbus.

15. The method of claim 11, further comprising preprocessing the image data to normalize illumination conditions and enhance contrast prior to identifying the limbus.

16. The method of claim 11, further comprising receiving patient-specific biometric data associated with the eye, wherein determining the safe insertion region is further based on the patient-specific biometric data, and wherein the patient-specific biometric data comprises one or more ocular measurements obtained from a preoperative diagnostic device.

17. The method of claim 11, wherein determining the safe insertion region comprises calculating a position of the safe insertion region based on a geometric relationship between a radius of the limbus and a radius of the eye.

18. The method of claim 11, wherein the one or more trocar insertion locations include at least three trocar insertion locations arranged at different angular positions within the safe insertion region.

19. The method of claim 11, wherein the video stream has a video frame rate of at least 30 frames per second (fps).

20. An ophthalmic microscope, comprising:
a floor stand;
a microscope optical head rotatably coupled to the floor stand;
a monoscopic microscope rotatably coupled to the microscope optical head;
a video camera attached to the monoscopic microscope, the video camera configured to generate a video stream comprising a sequence of video frames, each video frame comprising image data of an eye of a patient;
a display; and
a processor coupled to the video camera and the display, the processor configured to:
receive, from the video camera, the video stream;
for each video frame in the sequence of video frames:
generate, based on the image data, a segmentation mask identifying a limbus of the eye;
determine, based on the segmentation mask, geometric characteristics of the limbus;
determine, based at least in part on the geometric characteristics of the limbus, a safe insertion region located posterior to the limbus;
determine one or more trocar insertion locations within the safe insertion region;
generate an overlay identifying the one or more trocar insertion locations; and
present, on the display, the overlay with the image data.
